(19) United States
(12) Patent Application Publication
(10) Pub. No.: US 2014/0329192 A1
(43) Pub. Date: Nov. 6, 2014

(54) Electronically Enabled Removable Dental Device

(71) Applicants: Lucie R. Kaskoun, La Jolla, CA (US); Kenneth Kaskoun, La Jolla, CA (US)

(72) Inventors: Lucie R. Kaskoun, La Jolla, CA (US); Kenneth Kaskoun, La Jolla, CA (US)

(21) Appl. No.: 13/783,259

(22) Filed: Mar. 2, 2013

(51) Int. Cl. A61C 7/08; A61B 5/08; A61C 19/045; A61C 19/06; A61B 5/01

(52) U.S. Cl. CPC A61C 7/08; A61C 19/063; A61B 5/01; A61C 19/045; A61B 5/082
USPC 433/6; 433/27; 433/29; 433/80; 433/2; 433/215

(57) ABSTRACT

A dental device is provided in which the dental device comprises electronic components for ornamental functionality as well as orthodontic functionality. The dental device comprises components for light, sound, and video display for patients with braces, or for recreational users.

### BACKGROUND OF THE INVENTION

Braces and dental correction devices are becoming more and more popular as even adults are wearing them to correct dental misalignment. Wearing them can sometimes be uncomfortable and socially awkward. Even with the advent of see through devices it can still be awkward for even adults to wear these in public since they can still be seen and may make it uncomfortable to talk with people. Braces and mouth pieces are something that people notice and look at, especially when they are new to the user. A need exists for dental devices that allow wearer to show individuality.

### SUMMARY OF THE INVENTION

The above forms of the present invention are illustrative only and are not intended to limit the scope of the present invention. A person having skill in the art will understand that various modifications can be made without departing from the spirit of the present invention.

This disclosure describes various embodiments of a removable dental fixture. One would be a removable fixture with active electronics. These fixtures may be used for making corrections to the teeth and the electronics could allow lighting, pictures, video and even sounds making them fun to wear and allowing for a unique advertisement opportunity.

One embodiment of this invention may include a small self-contained electrical module that would include a battery or energy source, a wireless connection and an LED. The device could be integrated into the removable dental fixture by adhesives or injected molded directly into plastic mouth piece. The lighting could then be turned on or off either manually by a switch or via and application on a remote device such as a smartphone. The light then could be directed around the mouth either by attaching a light guide or in the case of molded mouth piece integrating the light guide into the mold.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an isometric view of the device according to one embodiment of the invention.

FIG. 2 shows an isometric view of the device imbedded within a mouthpiece according to one embodiment of the invention.

FIG. 3 shows a top view of the embodiment of the invention.

FIG. 4 shows a circuit diagram according to one embodiment of the invention.

### DETAILED DESCRIPTION OF THE INVENTION

While certain embodiments have been provided and described herein, it will be readily apparent to a person skilled in the art that such embodiments are provided by way of example only, It should be understood that various alternatives to the embodiments described herein may be employed, and are part of the invention described herein.

Provided herein is a dental device comprising an electrical module, energy source, and at least one of the following: a monitoring module, an audio/visual (A/V) module, and a fluid dispenser.

In some embodiments, the device further comprises fixtures to align and straighten teeth. These fixtures can comprise metal brackets to force teeth to grow or move in a certain direction, or a plastic mold to hold teeth to grow or move in a certain direction.

In some embodiments, the device is removably attached from an existing dental brace or a plastic mouth piece. In some embodiments, the device is attached by adhesives. In some embodiments, the device is injection molded directly into a plastic mouth piece. In some embodiments, the plastic mouth piece is Invisalign Clear Braces. In some embodiments, the existing dental brace is American Orthodontics orthodontic braces.

Fluids from the mouth can enter the electrical module and cause damage to both the device and the user. In some embodiments, the electrical module is hermetically sealed for electrical and chemical insulation. The device can be hermetically sealed by installing the device into a self-contained unit. The device can also be hermetically sealed by molding directly into a mouth piece.

In some embodiments, the energy source is a small rechargeable battery or capacitor. In further embodiments, the rechargeable battery or capacitor is charged by kinetic energy or winding a mechanical spring. Alternatively, for non-limiting purposes, the rechargeable battery or capacitor can be charged by kinetic energy derived from chewing, opening and closing the mouth, or talking. In other embodiments, the rechargeable battery or capacitor is charged by inductive coupling.

In some embodiments, the A/V module further comprises at least one of: a projector, light display, speaker, video recorder, and microphone. The projector can project an image or video into the user's mouth by either direct projection or reflecting the projection onto small mirrors or lenses fixed at certain points along the device. The projector can also project an image or video onto the front of the user's teeth by either direct projection or reflecting the projection onto small mirrors or lenses fixed at certain points along the device.

In some embodiments, the light display is a Light Emitting Diode (LED) or a small array of LED's that display various colors or images within the user's mouth. These colors can be controlled by a manual or wireless control located in the electrical module. The user can also issue voice commands in which a processor in the electrical module directs the device to change the color of the light display according to the voice command.

In some embodiments, the light display is a light guide fixture that can be attached to existing dental braces or directly molded into the plastic of a mouth piece. The light guide fixture can be a fiber optic guide. The light guide fixture can connect to certain embodiments of existing dental braces in which the light guide fixture is attached to the brackets of the dental braces tooth by tooth.

In some embodiments, the speaker is configured to play at least one of the following: music, sound recordings, sound bites, and projected audio.

In some embodiments, the A/V module further comprises a microphone used to record or receive audio. The microphone can also record audio onto a memory storage device found in the dental device.

In some embodiments, the A/V module further comprises a video recorder used to record video. The video recorder can record video from within the mouth, as well as outside the mouth. The video recorder can also record video onto a memory storage device found in the dental device.

In some embodiments, the electrical module further comprises a processor configured to respond to voice commands received by the microphone. The voice commands can be used to adjust the volume of speech of the user.

In some embodiments, the A/V module emits an image, video, or sound for advertising purposes. In some embodiments, a professional or collegiate sports team will purchase advertising space on the user's teeth. Further, in such embodiments, the advertising space will be used for displaying a slogan. Advertising space can be used to offset or defer the cost of the dental work.

In some embodiments, the monitoring module further comprises sensors to monitor health attributes within the mouth. The sensors comprise at least one of the following: a temperature sensor to measure the temperature within the mouth, a movement sensor to detect or measure movement of the teeth or jaw, and a gas sensor to detect or measure the level of a certain gas within the mouth. In some embodiments, the temperature sensor is configured to detect a fever. In some embodiments, the movement sensor is configured to detect or measure teeth grinding.

In some embodiments, the gas sensor is configured to detect the level of sulfur within the mouth which can indicate bad breath. In some embodiments, the gas sensor is used to detect or measure the level of alcohol within the mouth which indicates how much alcohol the user has consumed. The device can then wirelessly inform the user of his/her alcohol content. The device could then make further suggestions on whether the user can further consume alcohol or whether the user can legally drive. This information can be monitored by law enforcement. The removable dental device could be used to enforce a court order not to drink or not to drink and drive.

In some embodiments, the sensors can be used to monitor breath can include measuring the level of volatile sulfur compounds (VSC's) such as hydrogen sulfide, methyl mercaptan, Allyl methyl sulfide, and dimethyl sulfide. This could also help to diagnose if the user suffers from halitosis. The sensors can also be used to diagnose for gum diseases such as gingivitis or periodontitis. The electronic module can also include the ability to counter bad breath by shooting mint spray or other breath freshening fluids from within the dental device.

In some embodiments with a sensor to monitor teeth grinding, the electronic module can include a movement detection sensor to monitor the user's sleep. This sensor can also include the ability to measure the degree of teeth grinding. This data can be transmitted every night to the electronic module in order to record the user's daily teeth grinding. A warning signal can also be emitted through the device's speaker to alert the user of teeth grinding.

In some embodiments, the electrical module further comprises manual control including a simple on/off switch. In some embodiments, the manual control is used to turn the device on/off, configure color display settings, configure image or video settings, configure audio settings or receive and monitor data from sensors.

In some embodiments, the electrical module further comprises a wireless control including, but not limited to, Bluetooth communication or other wireless signal. The wireless control can be controlled by a tablet or smart phone that is used to turn the device on/off, configure color display settings, configure image or video settings, configure audio settings, or receive and monitor data from sensors. An application can be written that would allow the module to be programmed, to automate color, turning the device on or off, loading of specific images, sounds or video. Further applications could be written to allow the module to read and monitor data from sensors which can then allow the user to control the device.

In some embodiments, the fluid dispenser is configured to dispense mouthwash, water, or teeth whitening solution.

Provided herein is a method of using a dental device comprising an electrical module, an energy source, and at least one of the following:
(a) a monitoring module,
(b) an audio/visual (A/V) module, and
(c) a fluid dispenser.

The above forms of the present invention are illustrative only and are not intended to limit the scope of the present invention. A person having skill in the art will understand that various modifications can be made without departing from the spirit of the present invention.

This disclosure describes various embodiments of a removable dental fixture. One would be a removable fixture with active electronics. These fixtures can be used for making corrections to the teeth and the electronics could allow lighting, pictures, video and even sounds making them fun to wear and allowing for a unique advertisement opportunity.

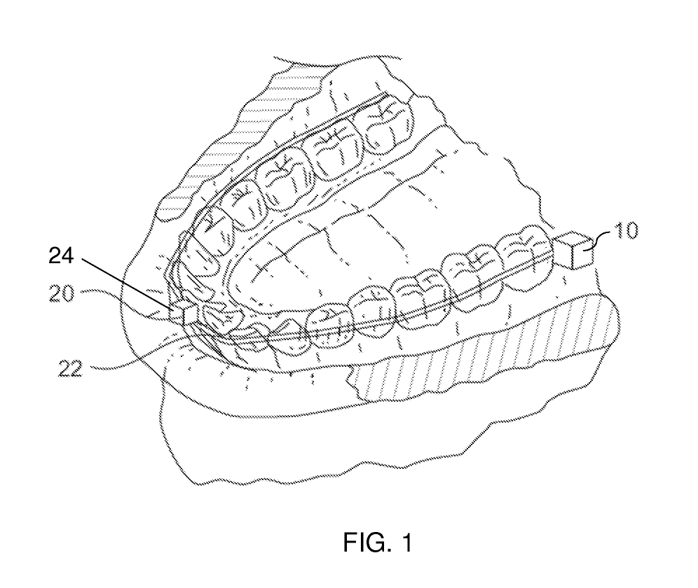

FIG. 1 shows an isometric view of a patient's lower teeth with the device fixed onto the teeth. The electronic module 10 is connected to the audio/visual (A/V) module 20. In some embodiments, the A/V module 20 can comprise a receiver, sensors, or a fluid dispenser. In this embodiment, the electronic module 10 sits behind the last molar. In other embodiments, the electronic module 10 sits on the inside of a row of teeth, or on the outside of a row of teeth. In this embodiment, the A/V module 20 contains a speaker 24 which projects audio from within the mouth. The A/V module 20 can be connected to a light guide 22 which emits a light along the teeth. In this embodiment, the light guide 22 wraps around the teeth and is fixed by adhesives or other physical attachment. In other embodiments, the light guide is installed in existing orthodontic braces by fixing the light guide to each individual bracket or by fixing the light guide along the long brace wire. In some embodiments, the A/V module 20 comprises a projector, which projects video or an image onto the front of the teeth.

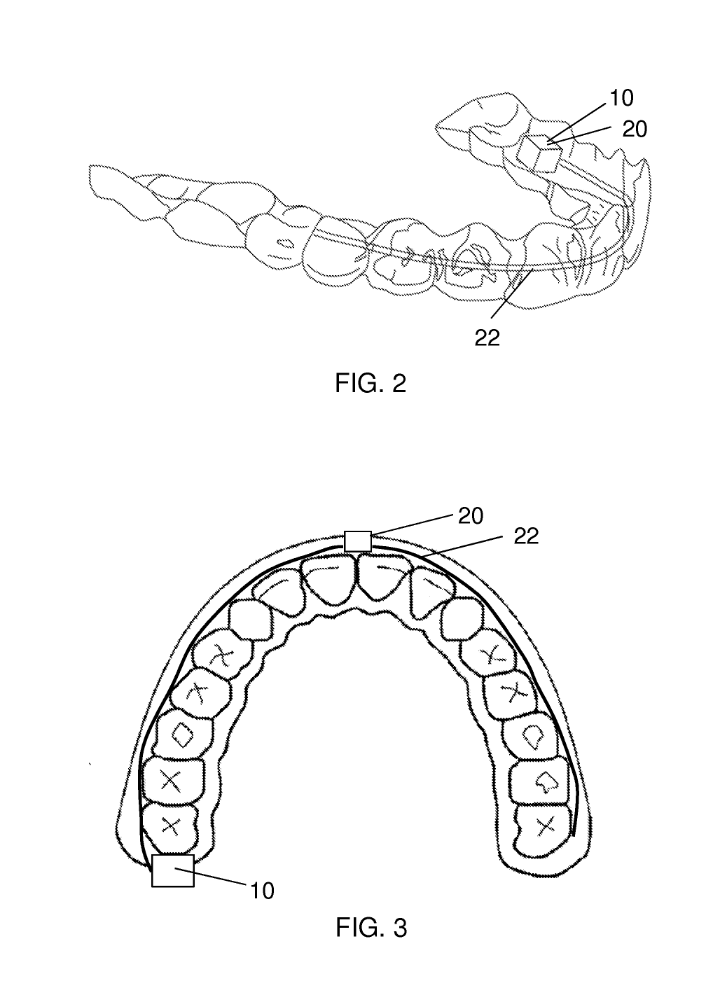

FIG. 2 shows an isometric view of the device imbedded in a mouthpiece. In this embodiment, the mouthpiece contains an electronic module 10, which also contains the A/V module 20. The electronic module is connected to a light guide 22 along the inside of the mouth piece. In other embodiments, the A/V module is fixed near the center of the dental device.

FIG. 3 shows a top view of the device portraying an electronic module 10 behind the teeth, an A/V module 20, and a light guide 22.

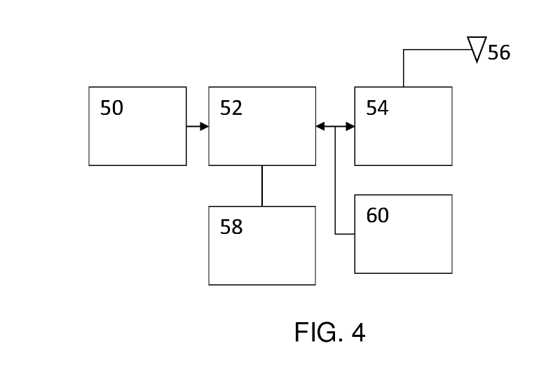

FIG. 4 shows a schematic of the electronic module. A power source 50 (or herein energy source) is configured to power the electronics. The power source 50 can either be a rechargeable battery or a rechargeable capacitor. The controls interface 52 is configured to control the module including manual interface such as switches and buttons. The radio 54 and antenna 56 are configured for wireless connection to transmit or receive information to an external device. The emitter/sensor 60 is controlled by the controls interface 52 which controls functions such as the light source, A/V, sensors, or fluid dispensing. The memory storage device 58 is configured to store data locally into the electronic module and also to store programmed functions into the electronic module.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A dental device comprising an electrical module, energy source, and at least one of the following:
(a) a monitoring module,
(b) an audio/visual (A/V) module, and
(c) a fluid dispenser.

2. The device of claim 1, further comprising fixtures to align and straighten teeth.

3. The device of claim 1, wherein the device is removably attached from an existing dental brace or corrective mouth piece.

4. The device of claim 1 wherein the electrical module is hermetically sealed for electrical and chemical insulation.

5. The device of claim 1 where the energy source is a small rechargeable battery or capacitor.

6. The device of claim 5 where the rechargeable battery or capacitor is charged by kinetic energy.

7. The device of claim 5 here the rechargeable battery or capacitor is charged by inductive coupling.

8. The device of claim 1, wherein the A/V module further comprises at least one of: a projector, light display, speaker, video recorder, and microphone.

9. The device of claim 8, wherein the projector projects an image or video in the user's mouth by either direct projection or reflecting the projection onto small mirrors or lenses fixed at certain points along the device.

10. The device of claim 9, wherein the projector projects an image or video onto the front of the user's teeth by either direct projection or reflecting the projection onto small mirrors or lenses fixed at certain points along the device.

11. The device of claim 8, wherein the light display is a Light Emitting Diode (LED) or a small array of LED's that display various colors or images within the user's mouth.

12. The device of claim 8, wherein the light display is a light guide fixture that can be attached to existing dental braces or directly molded into the plastic of a mouth piece.

13. The device of claim 8, wherein the speaker is configured to play at least one of he following: music, sound recordings, sound bites, and projected audio.

14. The device of claim 8, wherein the microphone is used to record or receive audio, and wherein the audio is stored onto a memory storage device found in the dental device.

15. The device of claim 8, wherein the video recorder is used to record or receive video, and wherein the video is stored onto a memory storage device found in the dental device.

16. The device of claim 1, further comprising a processor configured to respond to voice commands received by the microphone.

17. The device of claim 1, wherein the A/V module emits an image, video, or sound for advertising purposes.

18. The device of claim 1 wherein the monitoring module further comprises sensors to monitor health attributes within the mouth.

19. The device of claim 17, wherein the sensors comprise at least one of the following:
a temperature sensor to measure the temperature within the mouth,
a movement sensor to detect or measure movement of the teeth or jaw, and
a gas sensor to detect or measure the level of a certain gas within the mouth.

20. The device of claim 18, wherein the gas sensor is configured to indicate bad breath.

21. The device of claim 18, wherein the gas sensor is used to detect or measure the level of alcohol consumption.

22. The device of claim 1, wherein the electrical module further comprises manual control that is used to turn the device on/off, configure color display settings, configure image or video settings, configure audio settings, or receive and monitor data from sensors.

23. The device of claim 1, wherein the electrical module further comprises a wireless control including, but not limited to, Bluetooth communication or other wireless signal.

24. The device of claim 22, wherein the wireless control is controlled by a tablet or smart phone that is used to turn the device on/off, configure color display settings, configure image or video settings, configure audio settings, or receive and monitor data from sensors.

25. The device of claim 1 wherein the fluid dispenser is configured to dispense mouthwash, water, or teeth whitening solution.

26. A method of using a dental device comprising an electrical module, an energy source, and at least one of the following:
(a) a monitoring module,
(b) an audio/visual (A/V) module, and
(c) a fluid dispenser.
